(19) United States
(12) Patent Application Publication
(10) Pub. No.: US 2022/0392566 A1
(43) Pub. Date: Dec. 8, 2022

(54) Distillation of MSA Embeddings to Folded Protein Structures using Graph Transformers

(71) Applicant: Massachusetts Institute of Technology, Cambridge, MA (US)

(72) Inventors: Pranam Chatterjee, Cambridge, MA (US); Allan S Costa, Boston, MA (US); Joseph M. Jacobson, Newton, MA (US); Raghava Manvith Ponnapati, Cambridge, MA (US)

(73) Assignee: Massachusetts Institute of Technology, Cambridge, MA (US)

(21) Appl. No.: 17/831,435

(22) Filed: Jun. 2, 2022

Related U.S. Application Data
(60) Provisional application No. 63/196,125, filed on Jun. 2, 2021.

(51) Int. Cl. G16B 15/20; G06F 30/20

(52) U.S. Cl. CPC G16B 15/20; G06F 30/20

(57) ABSTRACT

An attention-based graph architecture that exploits MSA Transformer embeddings to directly produce models of three-dimensional folded structures from protein sequences includes a method and system for augmenting the protein sequence to obtain multiple sequence alignments, producing enriched individual and pairwise embeddings from the multiple sequence alignments using an MSA-Transformer, extracting relevant features and structure latent states from the enriched individual and pairwise embeddings for use by a downstream graph transformer, assigning individual and pairwise embeddings to nodes and edges, respectively, using the downstream graph transformer to operate on node representations through an attention-based mechanism that considers pairwise edge attributes to obtain final node encodings, and projecting the final node encodings to form the computer-modeled folded protein structure. An induced distogram of the computer-modeled folded protein structure may be computed.

### RELATED APPLICATIONS

This application claims the benefit of U.S. Provisional Application Ser. No. 63/196,125, filed Jun. 2, 2021, the entire disclosure of which is herein incorporated by reference.

### FIELD OF THE TECHNOLOGY

The present invention relates to protein structure modeling and, in particular, to a graph architecture that employs MSA transformer embeddings to produce models of three-dimensional folded structures from protein sequences.

### BACKGROUND

Determining the structure of proteins has been a long-standing goal in biology. Language models have recently been deployed to capture the evolutionary semantics of protein sequences. Enriched with multiple sequence alignments (MSA), these models can be employed to encode protein tertiary structure.

Elucidating protein structure is critical for understanding protein function. However, structure determination via experimental methods, such as x-ray crystallography [Smyth, M. S., “X ray crystallography”, Molecular Pathology, 53(1):8-14, 2000] or cryogenic electron microscopy (cryo-EM) [Murata, K. and Wolf, M., “Cryo-electron microscopy for structural analysis of dynamic biological macromolecules”, Biochimica et Biophysica Acta (BBA)—General Subjects, 1862(2):324-334, 2018], is a time-consuming, difficult, and expensive task. Classical modeling methods have attempted to solve this task in silico, but have been found to be computationally prohibitive [Rohl, C. A., Strauss, C. E., Misura, K. M., and Baker, D., “Protein structure prediction using Rosetta”, Methods in Enzymology, pages 66-93, Elsevier, 2004; Hollingsworth, S. A. and Dror, R. O., “Molecular dynamics simulation for all”, Neuron, 99(6):1129-1143, 2018; Wang, S., Li, W., Zhang, R., Liu, S., and Xu, J., “CoinFold: a web server for protein contact prediction and contact-assisted protein folding”, Nucleic Acids Research, 44(W1):W361-W366, 2016]. Recently, machine learning approaches have been deployed to harvest available structural data and efficiently map sequence-to-structure [Yang, J., Anishchenko, I., Park, H., Peng, Z., Ovchinnikov, S., and Baker, D., “Improved protein structure prediction using predicted interresidue orientations”, Proceedings of the National Academy of Sciences, 117(3):1496-1503, 2020; Senior, A. W., Evans, R., Jumper, J., Kirkpatrick, J., Sifre, L., Green, T., Qin, C., Židek, A., Nelson, A. W. R., Bridgland, A., Penedones, H., Petersen, S., Simonyan, K., Crossan, S., Kohli, P., Jones, D. T., Silver, D., Kavukcuoglu, K., and Hassabis, D., “Improved protein structure prediction using potentials from deep learning”, Nature, 577(7792):706-710, [2020].

Transformer models are sequence-to-sequence architectures that have been shown to capture the contextual semantics of words [Vaswani, A., Shazeer, N., Parmar, N., Uszkoreit, J., Jones, L., Gomez, A. N., Kaiser, L., and Polosukhin, I., “Attention is all you need”, Advances in neural information processing systems, 30, 2017] and have been widely deployed as language models [Devlin, J., Chang, M.-W., Lee, K., and Toutanova, K., “Bert: Pre-training of deep bidirectional transformers for language understanding”, NAACL HLT 2019 Conference of the North American Chapter of the Association for Computational Linguistics: Human Language Technologies—Proceedings of the Conference, 1: 4171-86, 2019; Brown, T. B., Mann, B., Ryder, N., Subbiah, M., Kaplan, J., Dhariwal, P., Neelakantan, A., Shyam, P., Sastry, G., Askell, A., Agarwal, S., Herbert-Voss, A., Krueger, G., Henighan, T., Child, R., Ramesh, A., Ziegler, D. M., Wu, J., Winter, C., Hesse, C., Chen, M., Sigler, E., Litwin, M., Gray, S., Chess, B., Clark, J., Berner, C., McCandlish, S., Radford, A., Sutskever, I., and Amodei, D., “Language models are few-shot learners”, Advances in neural information processing systems, 33, 1877-1901, 2020]. The sequential structure of proteins, imposed by the central dogma of molecular biology, along with their hierarchical semantics, as developed through Darwinian evolution, makes them a natural target for language modeling.

Recently, transformers have been deployed to learn protein sequence distributions and generate latent embeddings that grasp relevant structure [Rives, A., Meier, J., Sercu, T., Goyal, S., Lin, Z., Liu, J., Guo, D., Ott, M., Zitnick, C. L., Ma, J., and Fergus, R., “Biological structure and function emerge from scaling unsupervised learning to 250 million protein sequences”, Proceedings of the National Academy of Sciences, 118(15):e2016239118, 2021; Elnaggar, A., Heinzinger, M., Dallago, C., Rehawi, G., Wang, Y., Jones, L., Gibbs, T., Feher, T., Angerer, C., Steinegger, M., Bhowmik, D., and Rost, B., “ProtTrans: Towards cracking the language of life's code through self-supervised learning”, bioRxiv, pp. 2020-0′7, 2020; Vig, J., Madani, A., Varshney, L. R., Xiong, C., Socher, R., and Rajani, N. F., “BERTology meets biology: Interpreting attention in protein language models”, arXiv preprint arXiv:2006.15222, [2020], most notably tertiary structural information [Rao, R. M., Meier, J., Sercu, T., Ovchinnikov, S., and Rives, A., “Transformer protein language models are unsupervised structure learners”, International Conference on Learning Representations, 2020]. Augmenting input sequences with their evolutionarily-related counterparts, in the form of a multiple sequence alignment (MSA), further strengthens the predictive power of these transformer architectures, as demonstrated by state-of-art contact prediction results [Rao, R., Liu, J., Verkuil, R., Meier, J., Canny, J. F., Abbeel, P., Sercu, T., and Rives, A., “MSA transformer”, International Conference on Machine Learning, pp. 8844-8856. PMLR, 2021].

### SUMMARY

In one aspect, the present invention includes an attention-based graph architecture that exploits MSA Transformer embeddings to directly produce models of three-dimensional folded structures from protein sequences. It is envisioned that this pipeline will provide a basis for efficient, end-to-end protein structure prediction.

In this invention, MSA Transformer embeddings within a geometric deep learning architecture are leveraged to directly map protein sequences to folded, three-dimensional structures. In contrast to existing architectures, point coordinates are directly estimated in a learned, canonical pose, which removes the dependency on classical methods for resolving distance maps and enables gradient passing for downstream tasks, such as side-chain prediction and protein refinement. Overall, the results provide a bridge to a complete, end-to-end folding pipeline.

In one aspect, the invention is a method for computer modelling of a three-dimensional folded protein structure based on a protein sequence by using a computer processor to augment the protein sequence to obtain multiple sequence alignments, produce enriched individual and pairwise embeddings from the multiple sequence alignments using an MSA-Transformer, extract relevant features and structure latent states from the enriched individual and pairwise embeddings for use by a downstream graph transformer, assign individual and pairwise embeddings to nodes and edges, respectively, use the downstream graph transformer, which operates on node representations through an attention-based mechanism that considers pairwise edge attributes, to obtain final node encodings, and project the final node encodings to form the computer-modeled folded protein structure. The method may further include computing an induced distogram of the computer-modeled folded protein structure. The method may also include storing any individual and pairwise embeddings that are from the original protein sequence.

In another aspect, the invention is a method for folding a protein sequence in silico using an attention-based graph transformer architecture that includes the steps of using the MSA transformer to produce information-dense embeddings from the protein sequence, producing initial node and edge hidden representations in a complete graph from the embeddings, using the attention-based graph transformer architecture to process and structure geometric information in order to obtain final node representations, and projecting the final node representations into Cartesian coordinates through a learnable transformation to obtain the folded protein sequence. The method may further include the step of calculating induced distance maps from the projected final node representations. The induced distance maps may be compared to ground truth counterparts in order to define the loss.

In a further aspect, the invention is a system for producing models of three-dimensional folded protein structures from protein sequences, comprising a computer processor or set of processors specially adapted for performing the steps of augmenting a protein sequence to obtain multiple sequence alignments, using an MSA-Transformer, to produce enriched individual and pairwise embeddings from the multiple sequence alignments, extracting relevant features and structure latent states from the enriched individual and pairwise embeddings, for use by a downstream graph transformer, assigning individual and pairwise embeddings to nodes and edges, respectively, using the downstream graph transformer to operator on node representations through an attention-based mechanism that considers pairwise edge attributes to obtain final node encodings, and projecting the final node encodings to form a model three-dimensional folded protein structure. The computer processor or set of processors of the system may be further specially adapted for performing the step of computing an induced distogram of the computer-modeled folded protein structure.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 depicts an overview of a sequence-to-structure pipeline utilizing the MSA-Transformer and a Graph Transformer, according to one aspect of the present invention.

FIGS. 2A and 2B present comparisons of predicted distograms and three-dimensional arrangement with their ground truth counterparts for samples from ESM Structural Split dataset (FIG. 2A) and CASP13 Free Modeling targets (FIG. 2B), according to one implementation of the present invention.

FIG. 3 depicts a qualitative assessment of model predictions for CASP13 free modeling targets, according to one application of the present invention.

### DETAILED DESCRIPTION

In the present invention, the protein folding problem is treated as a graph optimization problem. Information-dense embeddings produced by the MSA Transformer [Rao, R., Liu, J., Verkuil, R., Meier, J., Canny, J. F., Abbeel, P., Sercu, T., and Rives, A., “MSA transformer”, International Conference on Machine Learning, pp. 8844-8856. PMLR, 2021] are harvested and then used to produce initial node and edge hidden representations in a complete graph. To process and structure geometric information, the attention-based architecture of the Graph Transformer is employed, as proposed by Shi et al. [Shi, Y., Huang, Z., Feng, S., Zhong, H., Wang, W., and Sun, Y., “Masked label prediction: Unified message passing model for semi-supervised classification”, arXiv preprint arXiv:2009.03509, 2021]. Final node representations are then projected into Cartesian coordinates through a learnable transformation, and the resulting induced distance maps are compared to their ground truth counterparts in order to define the loss for training.

MSA Transformer Data Augmentation

The MSA Transformer is an unsupervised protein language model that produces information-rich residue embeddings [Rao, R., Liu, J., Verkuil, R., Meier, J., Canny, J. F., Abbeel, P., Sercu, T., and Rives, A., “MSA transformer”, International Conference on Machine Learning, pp. 8844-8856. PMLR, 2021]. In contrast to other protein language models, it operates on two dimensional inputs consisting of a length-N query sequence along with its MSA sequences. It utilizes an Axial Transformer [Ho, J., Kalchbrenner, N., Weissenborn, D., and Salimans, T., “Axial attention in multidimensional transformers”, arXiv preprint arXiv:1912.12180, 2019] as an efficient attention-based architecture for performing computation on its layers' O(N·S) representations, where S is the total number of input MSA sequences.

In a preferred embodiment, the present invention operates on graph features distilled from MSA Transformer encodings. Last-layer residue embeddings capture individual and contextual residue properties. Similarly, the vector formed by pairwise attention scores at each layer and head captures attentive interactions between residue pairs. The richness of information present at these vectors has been previously demonstrated in state-of-the-art contact prediction [Rao, R., Liu, J., Verkuil, R., Meier, J., Canny, J. F., Abbeel, P., Sercu, T., and Rives, A., “MSA transformer”, International Conference on Machine Learning, pp. 8844-8856. PMLR, 2021]. The present invention extends those individual and pairwise embeddings to node and edge representations, demonstrating that learning over the resulting graph can resolve a protein's three-dimensional structure.

One particular implementation of the invention employs the 100 million parameter-sized ESM-MSA-1 model [Rao, R., Liu, J., Verkuil, R., Meier, J., Canny, J. F., Abbeel, P., Sercu, T., and Rives, A., “MSA transformer”, International Conference on Machine Learning, pp. 8844-8856. PMLR, 2021], which was trained on 26 million MSAs queried from UniRef50 and sourced from UniClust30. ESM-MSA-1 produces N residue embeddings, hᵢ*∈ [symbol] ⁷⁶⁸, and N×N attention score traces, hᵢⱼ*∈ [symbol] ¹⁴⁴, for each input sequence. Since the MSA Transformer is computationally expensive to evaluate for large S, even in the context of inference, the encodings were precomputed and made readily available for training. This implementation uses S=64, stored residue embeddings {hᵢ*}, and attention score traces, {hᵢⱼ*}^{j>i} for each query sequence.

For training and validation, the ESM Structural Split [Rives, A., Meier, J., Sercu, T., Goyal, S., Lin, Z., Liu, J., Guo, D., Ott, M., Zitnick, C. L., Ma, J., and Fergus, R., “Biological structure and function emerge from scaling unsupervised learning to 250 million protein sequences”, Proceedings of the National Academy of Sciences, 118(15):e2016239118, 2021] was used, which builds upon trRosetta's training dataset [Yang, J., Anishchenko, I., Park, H., Peng, Z., Ovchinnikov, S., and Baker, D., “Improved protein structure prediction using predicted interresidue orientations”, Proceedings of the National Academy of Sciences, 117(3):1496-1503, 2020]. To overcome the bottleneck associated with reading large encodings directly from the file system, the splits were fixed to the first superfamily split, as specified in Rives, et al., and its MSA Transformer encodings were serialized into tar shards. A virtual layer of data shuffling was added through the WebDataset framework [Aizman, A., Maltby, G., and Breuel, T., “High performance i/o for large scale deep learning”, IEEE International Conference on Big Data (Big Data), 5965-5967, 2019]. The resulting dataset of graph features has 0.25 TB.

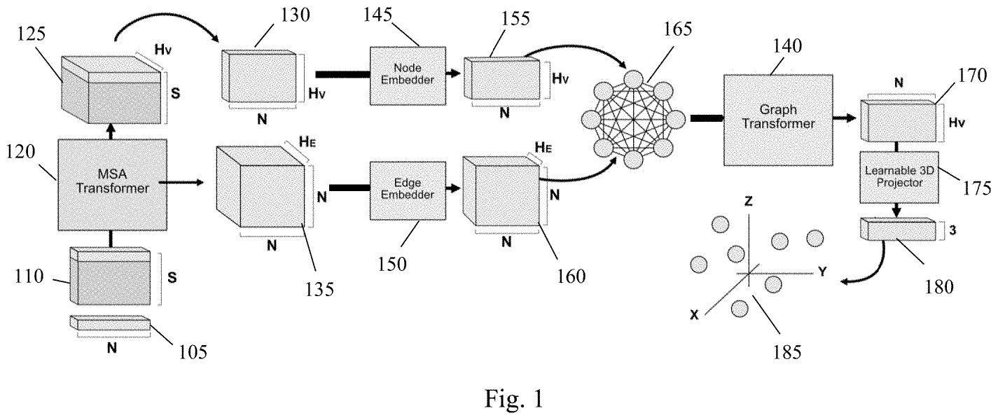

FIG. 1 depicts an overview of a sequence-to-structure pipeline utilizing the MSA-Transformer and a Graph Transformer. As shown in FIG. 1, a length-N protein sequence 105 is augmented 110 to S of its MSA. MSA-Transformer 120 operates over this token matrix 125 to produce enriched individual 130 and pairwise 135 embeddings. Those embeddings that are from the original query sequence are stored. Deep neural networks then extract relevant features and structure latent states for downstream graph transformer 140. Individual 130 and pairwise 135 embeddings are assigned 145, 150 to nodes 155 and edges 160, respectively. Graph transformer 140 operates on node representations 165 through an attention-based mechanism that considers pairwise edge attributes. Final node encodings 170 are projected 175 directly to [symbol] ³ 180, and the induced distogram 185 is computed for the loss.

Graph Building

In a preferred embodiment, a protein is treated as an attributed complete graph. H_{V} and H_{E} are the dimensionalities of node and edge representations, respectively. These attributes are extracted from MSA-Transformer embeddings through standard deep neural networks:

hᵢ=σ(W_{E}^{(D}^{_{V}}⁾ . . . (σ(W_{V}⁽⁰⁾hᵢ*)) . . . )

hᵢⱼ=σ(W_{D}^{(D}^{_{E}}⁾ . . . (σ(W_{E}⁽⁰⁾hᵢⱼ*)) . . . )

where hᵢ∈ [symbol] ^{H}^{_{V}}, hᵢⱼ∈ [symbol] ^{H}^{_{E}}, σ(⋅) is a ReLU nonlinearity, and D_{V} and D_{E} are the depths of node and edge information extractors, respectively. W denotes dense learnable parameters, and here and in the following equations bias terms are omitted.

Graph Transformer

The Graph Transformer used in the preferred embodiment was introduced in Shi et al. [Shi, Y., Huang, Z., Feng, S., Zhong, H., Wang, W., and Sun, Y., “Masked label prediction: Unified message passing model for semi-supervised classification”, arXiv preprint arXiv:2009.03509, 2021] in order to incorporate edge features directly into graph attention. This is possible by directly summing transformations of edge attributes to the original keys and values of the attention mechanism. The present invention approaches protein folding with a variation of this architecture. Considering layer l node hidden states, {hᵢ^{l}}, and similarly learned edge latent states, {eᵢⱼ}, if C attention heads are employed, a layer update can be written as

$${h}_{i}^{l + 1} = {W}_{R}^{( l )} {h}_{i}^{l} + σ ( {W}_{A}^{( l )} {⊕}_{c = 1}^{C} {\sum }_{j ∈ 𝒩 ( i )} {α}_{i , j}^{( l , c )} ( {v}_{j}^{( l , c )} + {e}_{ij}^{( c )} ) )$$

where ⊕ denotes concatenation, and W_{A}^{(l)} and W_{R}^{(l)} are learnable projections. As in the original architecture, batch normalization is applied to each layer. The attention scores αᵢⱼ^{(l,c)}, node values vⱼ^{(l,c)} and edge values eᵢⱼ^{(c)} are obtained from learnable transformations of the original node hidden states and edge attributes:

qᵢ^{(l,c)}=W_{q}^{(l,c)}hᵢ^{(l)} kᵢ^{(l,c)}=Wₖ^{(l,c)}hᵢ^{(l)}

vᵢ^{(l,c)}=Wᵥ^{(l,c)}hᵢ^{(l)} eᵢⱼ^{(c)}=Wₑ^{(c)}hᵢⱼ

The attention scores are normalized according to graph attention:

$${\overline{α}}_{i , j}^{( l , c )} = {( {q}_{i}^{( l , c )} )}^{T} ( {k}_{i}^{( l , c )} + {e}_{i j}^{( c )} ) {α}_{i , j}^{( l , c )} = \frac{exp [ {\overline{α}}_{i , j}^{( l , c )} ]}{{\sum }_{u ∈ 𝒩 ( i )} exp [ {\overline{α}}_{i , u}^{( l , c )} ]}$$

To hold computational costs roughly constant, {qᵢ^{c}, vᵢ^{c}, kᵢ^{c}, eᵢⱼ^{c}}∈ [symbol] ^{H}^{_{V}}^{/C}, as in standard Transformer architectures.

Cartesian Projection and Loss

In a preferred embodiment, a predictor is trained to recover coordinates of each residue in a learned canonical pose:

Xᵢ=W_{X}hᵢ^{(L)}

where Xᵢ∈ [symbol] ³. To train the network, a distogram-based loss function is used on the resulting distance map. \hat{D}ᵢⱼ=∥Xᵢ−Xⱼ∥₂ is the induced Euclidean distance between the Cartesian projections of nodes i and j, and Dᵢⱼ is the ground truth distance. The loss is based on the L₁-norm of the difference between those values:

$$ℒ = \frac{1}{{N}^{2}} {\sum }_{i}^{N} {\sum }_{j}^{N} {‖ {\hat{D}}_{i j} - {D}_{i j} ‖}_{1}$$

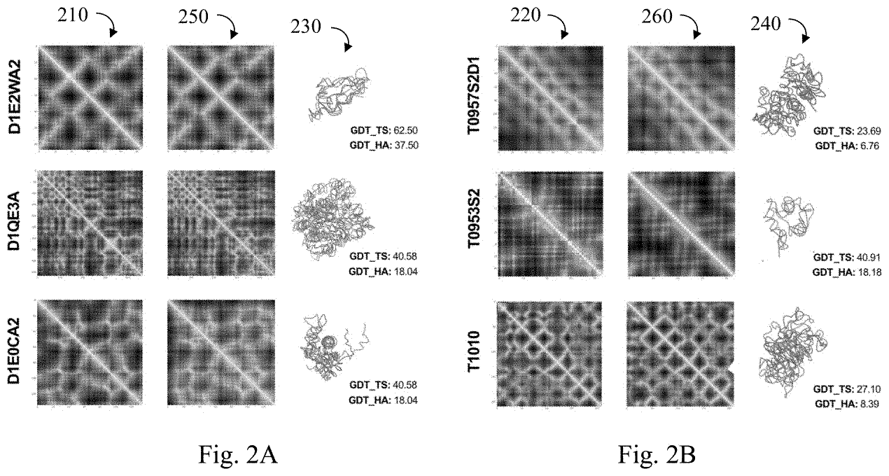

FIGS. 2A and 2B depict example comparisons of predicted distograms 210, 220 and three-dimensional arrangements 230, 240 with their respective ground truth counterparts 250, 260 for samples from the ESM Structural Split dataset (FIG. 2A) and CASP13 free modeling targets (FIG. 2B). In FIGS. 2A and 2B, for each prediction-ground truth pair, a PDB name is indicated on the left. Aligned Cα traces and metrics are indicated on the right. In three-dimensional arrangements 230, 240, blue traces denote model predictions and red traces denote ground truth. Traces were produced by fitting splines to the sequence of predicted Cα coordinates.

Model Training

To optimize the trained model, a shallow random hyperparameter search for H_{V}∈{32, 64, 128, 256}, H_{E}∈{32, 64, 128}, L∈{3, 6, 10, 15}, C∈{1,2,4} was performed. The Adam Optimizer was utilized, with lr∈{1×10⁻³, 3×10⁻⁴, 1×10⁻⁴, 3×10⁻⁵, 1×10⁻⁵}. Variations of the loss function were also tested, testing the MSE loss and weighted versions of L₁ and MSE for batch sizes B∈{10, 15, 30}.

To handle GPU memory constraints, gradient checkpointing was employed at each Graph Transformer layer. Models were trained in parallel on NVIDIA V100s provided by the MIT SuperCloud HPC [Reuther, A., Kepner, J., Byun, C., Samsi, S., Arcand, W., Bestor, D., Bergeron, B., Gadepally, V., Houle, M., Hubbell, M., et al., “Interactive supercomputing on 40,000 cores for machine learning and data analysis”, 2018 IEEE High Performance extreme Computing Conference (HPEC), pages 1-6, 2018].

In total, 40 search training runs were performed, with a maximum of 70 epochs and an early stop with a patience of 3 for validation loss. The best model trained for 17 hours without registering early stopping. With H_{V}=H_{E}=64, L=10, and C=1, this model only possesses a total of 382K parameters. Using lr=3×10⁴ and B=30, as well as an L₁ loss, [symbol] ᵥₐₗ=2.25 and GDT_TSᵥₐₗ=40.58 was achieved.

CASP13 Evaluation

To investigate the generalization of the model of the invention, it was evaluated on the free modeling targets from the 13th edition of the Critical Assessment of Protein Structure Prediction (CASP13). The model was benchmarked against the performance of the current state-of-the-art public architecture: trRosetta [Yang, J., Anishchenko, I., Park, H., Peng, Z., Ovchinnikov, S., and Baker, D., “Improved protein structure prediction using predicted interresidue orientations”, Proceedings of the National Academy of Sciences, 117(3):1496-1503, 2020]. trRosetta considers a sequence's MSA to predict distance probability volumes as well as relevant interresidue orientations. In contrast to the present invention, trRosetta relies on restraints derived from the predicted distance and orientations for downstream Rosetta minimization protocols [Rohl, C. A., Strauss, C. E., Misura, K. M., and Baker, D., “Protein structure prediction using Rosetta”, Methods in Enzymology, pages 66-93, Elsevier, 2004]. For each distance, trRosetta's best prediction is considered to be its expected value or its maximum likelihood estimate. dRMSD (distogram RMSD) between predicted distances and ground truth was utilized as the evaluation metric. To make a direct comparison, only distances that lie within trRosetta's binning range (2-20 Å) were considered.

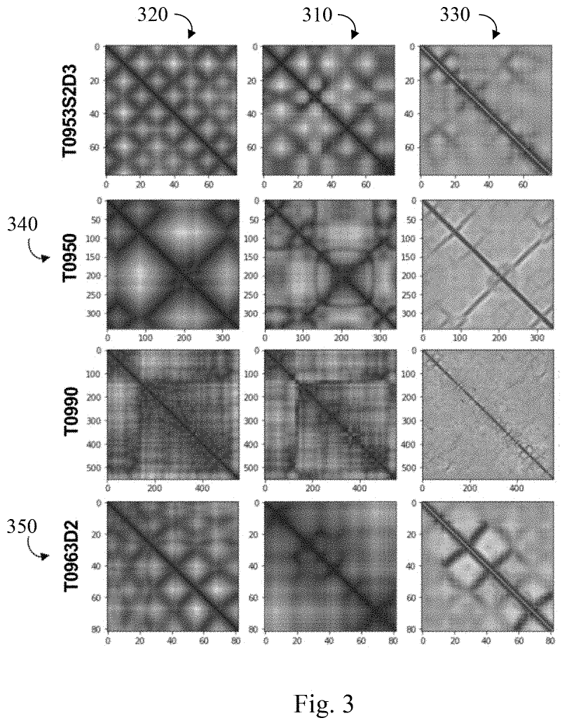

FIG. 3 presents an example qualitative assessment of model predictions 310 for CASP13 free modeling targets versus ground truth 320 and trRosetta 330. Note that the model is able to capture long range interactions, whereas trRosetta by construction is limited to short range dependencies. T0950 340 and T0963D2 350, in particular, are examples of challenging reconstructions for the network.

Table 1 presents a comparison of CASP13 Free Modeling benchmarks of dRMSD for the architecture of the present invention's induced distances and trRosetta's expectation and argmax distances, against ground truth, considering only distances that lie within trRosetta's binning range.

TABLE 1

| | T0987 | T0969 | T0955d1 | T0998 |
|---|---|---|---|---|
| Graph Transformer | 3.722 | 3.080 | 5.346 | 3.476 |
| trRosetta (argmax) | 2.135 | 1.583 | 2.400 | 1.482 |
| trRosetta | 1.638 | 1.288 | 2.160 | 1.247 |
| (expectation) | | | | |

| | T0990 | T0958d1 | T0968s2d1 | T0963d2 |
|---|---|---|---|---|
| Graph Transformer | 3.017 | 2.886 | 3.380 | 7.853 |
| trRosetta (argmax) | 1.356 | 1.947 | 1.927 | 4.039 |
| trRosetta | 1.078 | 1.796 | 1.695 | 2.982 |
| (expectation) | | | | |

| | T0953s2d3 | T1010 | T0968s1d1 | T0957s2d1 |
|---|---|---|---|---|
| Graph Transformer | 5.404 | 4.002 | 3.905 | 2.559 |
| trRosetta (argmax) | 4.647 | 2.048 | 2.226 | 1.700 |
| trRosetta | 3.681 | 1.531 | 1.797 | 1.492 |
| (expectation) | | | | |

| | T0950 | T0953s1 | T0953s2 | T1022s1 |
|---|---|---|---|---|
| Graph Transformer | 3.392 | 2.698 | 4.158 | 2.604 |
| trRosetta (argmax) | 1.542 | 1.897 | 3.868 | 1.665 |
| trRosetta | 1.148 | 1.618 | 3.144 | 1.433 |
| (expectation) | | | | |

These results demonstrate that the Graph Transformer model, despite its size, is competitive to trRosetta's estimates. It is worth noting that the architecture of the present invention resolves backbone structure as its main output and uniquely and deterministically produces distances, whereas trRosetta operates within a probabilistic domain that does not need three-dimensional resolution. These results thus suggest potential for improved predictive capability with larger model capacity and downstream protein refinement.

Importantly, in contrast to existing approaches, the present invention is highly computationally efficient and can be performed using a fairly small cluster of machines.

The present invention revisits the protein folding problem and highlights the role of unsupervised language models in providing a meaningful basis for the sequence-to-structure prediction task. It provides a strategy to encapsulate MSA Transformer embeddings and attention traces in a geometric framework, and formalize a graph learning pipeline to reason positional information.

Overall, the results demonstrate the remarkably expressive power of language models and, in particular, of MSA-augmented architectures. To demonstrate a versatile bridge between sequence and three-dimensional structure, a downstream model was trained to produce C-traces which, before any refinement is performed, induce distograms with high similarity to ground truth.

The model, in its currently preferred embodiment, tackles only a step of the protein structure prediction problem. With only 382K parameters, it serves as a fast and scalable solution to resolving the position of protein backbones. Furthermore, it extends learning beyond distogram prediction and provides a natural foundation for downstream tasks, such as side chain prediction and protein refinement. It is hypothesized that, by increasing model capacity, dataset size, and training time, the model's predictive capability can improve significantly.

The present invention builds upon recent groundbreaking work in protein representation learning and protein language modeling. The integration of diverse network architectures and pretrained models, as demonstrated by the present invention, will enable the eventual efficient solution of the protein structure prediction problem.

At least the following aspects, implementations, modifications, and applications of the described technology are contemplated by the inventors and are considered to be aspects of the presently claimed invention:

(1) Methods of folding a protein sequence in silico employing attention-based graph transformer architectures.

(2) Refinement of structures determined via the method of (1), utilizing physical and molecular simulations, in silico relaxation, and 3D roto-translation equivariant attention networks (SE3 transformers), according to techniques known in the art of the invention.

Some aspects of the invention incorporate methodologies that are disclosed via reference to one or more cited references. These methodologies are described in detail in one or more of the cited references, all of which are incorporated by reference herein.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention

What is claimed is:

1. A method for computer modelling of a three-dimensional folded protein structure based on a protein sequence, comprising:
using a computer processor, performing the steps of:
augmenting the protein sequence to obtain multiple sequence alignments;
using an MSA-Transformer, producing enriched individual and pairwise embeddings from the multiple sequence alignments;
extracting, from the enriched individual and pairwise embeddings, relevant features and structure latent states for use by a downstream graph transformer;
assigning individual and pairwise embeddings to nodes and edges, respectively;
using the downstream graph transformer, operating on node representations through an attention-based mechanism that considers pairwise edge attributes to obtain final node encodings; and
projecting the final node encodings to form the computer-modeled folded protein structure.

2. The method of claim 1, further comprising computing an induced distogram of the computer-modeled folded protein structure.

3. The method of claim 1, further comprising storing any individual and pairwise embeddings that are from the original protein sequence.

4. A method for folding a protein sequence in silico using an attention-based graph transformer architecture, comprising:
using the MSA transformer, producing information-dense embeddings from the protein sequence;
from the embeddings, producing initial node and edge hidden representations in a complete graph;
using the attention-based graph transformer architecture, processing and structuring geometric information, to obtain final node representations; and
projecting the final node representations into Cartesian coordinates through a learnable transformation to obtain the folded protein sequence.

5. The method of claim 4, further comprising calculating induced distance maps from the projected final node representations.

6. The method of claim 5, further comprising comparing the induced distance maps to ground truth counterparts in order to define the loss.

7. A system for producing models of three-dimensional folded protein structures from protein sequences, comprising a computer processor or set of processors specially adapted for performing the steps of:
augmenting a protein sequence to obtain multiple sequence alignments;
using an MSA-Transformer, producing enriched individual and pairwise embeddings from the multiple sequence alignments;
extracting, from the enriched individual and pairwise embeddings, relevant features and structure latent states for use by a downstream graph transformer;
assigning individual and pairwise embeddings to nodes and edges, respectively;
using the downstream graph transformer, operating on node representations through an attention-based mechanism that considers pairwise edge attributes to obtain final node encodings; and
projecting the final node encodings to form a model three-dimensional folded protein structure.

8. The system of claim 7, wherein the computer processor or set of processors is further specially adapted for performing the step of computing an induced distogram of the computer-modeled folded protein structure.
